(19) United States
(12) Patent Application Publication
(10) Pub. No.: US 2024/0077486 A1
(43) Pub. Date: Mar. 7, 2024

(54) Flow Cytometry Platform for the Detection of Glycosylated Proteins in a Clinical Sample

(71) Applicant: FlowMetric Life Sciences, Inc., Doylestown, PA (US)

(72) Inventors: Renold Julius Capocasale, Mount Laurel, NJ (US); Patrick Romano, Doylestown, PA (US); Julie Ann Bick, Easton, PA (US)

(21) Appl. No.: 18/270,871

(22) PCT Filed: Jan. 5, 2022
(86) PCT No.: PCT/US22/11295

§ 371 Date: Jul. 5, 2023

Related U.S. Application Data
(60) Provisional application No. 63/134,318, filed on Jan. 6, 2021.

(51) Int. Cl. G01N 33/574; C07K 14/42; G01N 33/543

(52) U.S. Cl. CPC G01N 33/57438; C07K 14/42; G01N 33/54353; G01N 2333/42; G01N 2400/02; G01N 2440/38

(57) ABSTRACT

Described herein is a flow cytometry platform for the detection of glycosylated proteins in a clinical sample, along with the use of a flow cytometry platform for early disease diagnoses, for example, hepatocellular carcinoma (HCC). The use of the flow cytometry platform described herein allows for multiplexing and quantification of two or more biomarkers associated with the disease, for example, alpha-feto protein (AFP), alfa-feto protein-L3 (AFP-L3), or alpha-L-fucosidase (AFU) for HCC patients.

### CROSS-REFERENCE TO RELATED APPLICATIONS

This application is the United States national phase of International Application No. PCT/US22/11295 filed Jan. 5, 2022, and claims priority to U.S. Provisional Application No. 63/134,318 filed Jan. 6, 2021, the disclosures of which are hereby incorporated by reference in their entireties.

### SEQUENCE LISTING

The Sequence Listing associated with this application was filed in electronic format via EFS-Web and is hereby incorporated by reference into the specification in its entirety. The name of the text file containing the Sequence Listing is 9255-1905360_ST25.txt. The size of the text file is 4,253 bytes, and the text file was created on Jan. 4, 2022.

### FIELD OF THE INVENTION

The present disclosure relates to an engineered lectin polypeptide having an amino acid sequence of SEQ ID NO. 1. The present disclosure further relates to a method of flow cytometry for detecting a glycosylation site in a sample including establishing a fluid stream, adding a sample having one or more glycosylated proteins to the fluid stream, selecting one or more probes and a detection molecule, and detecting the one or more glycosylated proteins by quantifying the detection molecule.

### SUMMARY OF THE INVENTION

Liver cancer is the fifth most common malignancy and the second most common cause of cancer-related death worldwide. The incidence of hepatocellular carcinoma (HCC) in the U.S. is increasing and is largely attributed to hepatitis C infection. While the survival of patients with almost all other types of cancer has improved over the last decade, the five-year survival of patients with HCC has remained at less than 10 percent. This poor outcome of patients with HCC is largely related to late detection and more than two-thirds of patients diagnosed have cancers that have progressed to advanced stages of disease. There are several factors behind this:firstly, HCC surveillance and diagnosis has been hampered by the lack of reliable biomarkers, and secondly by the poor sensitivity and reliability of the current enzyme linked immunosorbent assay (ELISA) based HCC diagnostic tests.

Alpha-Fetoprotein (AFP) is currently considered to be the most useful biomarker for Hepatocellular Carcinoma (HCC) evaluation. AFP is a glycoprotein with a molecular weight of about 70 kDa that can be produced during fetal and neonatal development by the liver, yolk sac, and in small concentrations, the gastrointestinal tract. Serum AFP reaches a maximal concentration of 3 g/L at weeks 12 to 16 of fetal life. Protein levels subsequently decrease rapidly, and thereafter only trace amounts are normally detected in serum. Abnormally high serum AFP concentrations can be correlated with the development and progression of several cancers, including, but not limited to, HCC. Total serum AFP can be distinguished into three different glycosylated isoforms, AFP-L1, AFP-L2, and AFP-L3-based on their binding capability to lectin Lens culinaris agglutinin (LCA). Increased serum level of AFP-L3 has been associated with poor liver function, and an overall higher tumor burden.

Health care professionals can measure AFP in a patient's blood. Typical AFP measurements are reported as nanograms per milliliter (ng/mL). The normal level for most healthy adults is between 0 and 8 ng/mL. Many factors, including cancer, liver disease (e.g., hepatitis or cirrhosis), as well as an injured liver that is healing, may increase AFP levels, and further testing may be required to obtain a proper diagnosis. High levels of AFP (e.g., 500 to 1,000 ng/mL) can be a sign of certain kinds of cancers. In patients with liver disease, an AFP level of more than 200 ng/mL may indicate progression to liver cancer. For patients with an increased AFP, but less than 200 ng/mL, health care professionals may perform a glycosylated isoform measurement of AFP termed an AFP-L3 measurement (also known as L3AFP). This type of measurement compares the amount of a specific glycosylated isoform of AFP (AFP-L3) to the total amount of AFP in a patient's blood. This type of comparison may help health care professionals diagnose and treat patients, especially in the context of a chronic liver disease (e.g., cirrhosis). An AFP-L3 measurement of 10% or more may suggest that a patient has an increased chance of developing liver cancer.

In addition to AFP as a useful biomarker for HCC evaluation, a number of endogenous proteins exist in two or more different isoforms that differ only in their pattern of glycosylation (i.e., glycosylated isoforms). As with AFP and its glycosylated isoforms being a useful biomarker for HCC, glycosylated isoforms of one or more proteins may be indicative of a disease or a disorder in a patient. There is therefore a need for testing systems capable of distinguishing between the glycosylated isoforms of various proteins. Any protein with post-translational glycosylation can potentially occur in different glycosylation isoforms. Glycosylated isoforms of various proteins are typically measured using antibodies to detect a glycosylation site(s) of the various proteins. However, the use of antibodies to distinguish between glycosylated isoforms of endogenous proteins is problematic and challenging, and the success rate in raising antibodies which bind specifically or preferentially to one particular glycosylated isoform of an endogenous glycosylated proteins is relatively low.

The determination of the relative concentrations of differentially glycosylated isoforms of an endogenous protein has been shown to be clinically important. Keir et al. discuss the abnormal relative abundance of the transferring glycosylated isoforms in patients with carbohydrate deficient glycoprotein syndromes or congenital disorder of glycosylation (Keir et al. Ann. Clin. Biochem. 36: 20-36, 1999). As such, clinically relevant proteins may exist in different glycosylated isoforms, including glycosylated markers for cancers and other disease. These proteins may include, but not limited to, alkaline phosphatase, alpha-fetoprotein, human chorionic gonadotropoin, and prion protein (CD230).

Accordingly, there is a need to provide an assay that can selectively distinguish one or more glycosylated isoforms of one or more proteins having various glycosylation profiles.

Flow cytometry is a highly sensitive analytical tool for measuring the presence of biomarkers on the surface or within cells. It is particularly well suited for the interrogation of immune cells from blood. However, by utilizing capture beads for specific serum solutes, flow cytometry can be adapted for the detection of serum-based biomarkers and their glycosylated isoforms (e.g., AFP). By using antibodies specifically targeting proteins of interest (e.g., AFP), capture beads can be used to determine total protein levels in serum. Further, the use of Aleuria aurantia lectin (AAL) or a Lens culinaris agglutinin (LCA) lectins specific for disease-associated glycosylated isoforms of proteins, can provide highly sensitive detection of glycosylated isoforms of proteins associated with the disease (e.g., fucosylated AFP3 can serve as an early diagnostic for HCC).

Flow cytometry is a technique for counting and examining small particles such as cells by suspending them in a stream of fluid and passing them by an electronic detection apparatus. It allows simultaneous multiparametric analysis of the physical and/or chemical characteristics of each individual particle or cell. Briefly, a beam of light (usually laser light) of a single wavelength is directed onto a hydrodynamically-focused stream of fluid. A number of detectors are aimed at the point where the stream passes through the light beam: one in line with the light beam (forward scatter), several in perpendicular position (side scatter) and at least one fluorescence detector. Each suspended cell passing through the light beam scatters the light in some way, and fluorescent molecules may be excited into emitting light at a longer wavelength than the light source. This combination of scattered and fluorescent light is recorded by detectors. The forward scatter correlates with the cell volume, while the side scatter depends upon the inner complexity of the cell. The data generated by flow-cytometers can be plotted in a single dimension to produce a histogram or in two-dimensional or three-dimensional plots. The regions on these plots can be sequentially separated, based on fluorescence intensity, by creating a series of subset extractions, termed “gates”. Specific gating protocols exist for diagnostic and clinical purposes.

As disclosed above, total serum AFP can be distinguished into three different glycosylated isoforms, AFP-L1, AFP-L2, and AFP-L3, based on their binding capability to AAL or LCA. Typically, AFP-L3 glycosylated isoforms are fucosylated at one or more alpha-1,6 fucosylation site(s). Increased serum level of AFP-L3 specifically has been associated with poor liver function, and an overall higher tumor burden. Accordingly, determining the percentage of AFP-L3 glycosylated isoforms that contain one or more alpha-1,6 fucosylation site(s) from the total AFP in serum could serve as a predictive marker for early detection of HCC. Further, one problem with using AFP alone as a reliable HCC biomarker is that 60%-80% of patients with HCC display elevated serum AFP, and high false-positive rates can make it difficult to distinguish early stage HCC from other disorders, such as acute hepatitis, cirrhosis, and certain gastrointestinal tumors. Therefore, for diagnostic accuracy for HCC, additional biomarkers are needed to complement AFP. One such biomarker is Alpha-1-fucosidase (AFU), a lysosomal enzyme that hydrolyzes the fucose glycosidic linkages of glycoprotein and glycolipids. AFU activity increases in the serum of HCC patients compared with that in the serum of healthy individuals, patients with cirrhosis and patients with chronic hepatitis. AFU level determination is useful in association with AFP in early diagnosis of HCC and could serve as a valuable supplementary to AFP. Clinical studies have indicated that HCC will develop within a few years in 82% of patients with liver cirrhosis if their serum AFU activity exceeds 700 nmol/mL/h, and importantly, the activity of AFU was reported to be elevated in 85% of patients at least 6 months before the detection of HCC by ultrasonography.

Other glycoproteins associated with disorders and considered potential targets for assay development in the present invention include, but not limited to, alpha-1-acid glycoprotein, alpha-1-antitrypsin, haptoglobin, thyroglobulin, prostate specific antigen, HEMPAS erythrocyte band 3 (associated with congenital dyserythropoietic anemia type II), PC-1 plasma-cell membrane glycoprotein, CD41 glycoprotein IIb, CD42b glycocalicin, CD43 leukocyte sialoglycoprotein, CD63 lysosomal-membrane-associated glycoprotein 3, CD66a biliary glycoprotein, CD66f pregnancy specific b1 glycoprotein, CD164 multi-glycosylated core protein 24, and the Cd235 glycophorin family.

An object of certain embodiments of the present disclosure is to provide an engineered lectin polypeptide having an amino acid sequence of SEQ ID NO. 1.

In some embodiments, the amino acid sequence of SEQ ID NO. 1 is:

MPTEFLYTSKIAAISWAATGGRQQRVYFQDLNGKIREAQRG
GDNPWTGGSSQNVIGEAKLFSPLAAVTWKSAQGIQIRVYCV
NKDNILSEFVYDGSKWITGNLGSVGVKVGSNSKLAALQWGG
SESAPPNIRVYYQKSNGSGSSIHEYVWSGKWTAGASFGSTVP
GTGIGATAIGPGRLRIYYQATDNKIREHCWDSNSWYVGGFSA
SASAGVSIAAISWGSTPNIRVYWQKGREELYEAAYGGSWNT
PGQIKDASRPTPSLPDTFIAANSSGNIDISVFFQASGVSLQQW
QWISGKGWSIGAVVPTGTPAGWLEHHHHHHHHHH.

In some embodiments, the lectin polypeptide may bind to one or more glyosylation sites of aprotein, wherein the protein is selected from the group consisting of alpha-feto protein (AFP), alfa-feto protein-L3 (AFP-L3), alpha-L-fucosidase (AFU), alpha-glucoside, basic-fibroblast growth factor (bFGF), glypican-3, alpha-1-fucosidase, gamma-glutamyl transferase, golgi phosphoprotein 2, transforming growth factor beta, tumor specific growth factor, and hepatocyte growth factor. In some embodiments, the one or more glycosylation sites of the protein is an alpha-1,6 fucosylation site, a L-fucopyranosyl site, an alpha 1-2 L-fucopyranosyl site, an alpha 1-3 L-fucopyranosyl site, or an alpha 1-4 L-fucopyranosyl site.

In further embodiments, the engineered lectin polypeptide may have a detection molecule wherein the detection molecule is selected from the group consisting of a capture antibody, a capture bead, a fluorophore and a combination thereof. In some embodiments, the capture bead has a size from about 5 microns to about 15 microns.

In some embodiments, the engineered lectin polypeptide is an Aleuria aurantia lectin (AAL) probe or a Lens culinaris agglutinin (LCA) probe. In some embodiments, the AAL probe may have three or more fucosylated oligosaccharide binding sites.

Another object of certain embodiments of the present disclosure is to provide a method of flow cytometry for detecting a glycosylation site in a sample. The method includes establishing a fluid stream. A sample having one or more glycosylated protein(s) is added to the fluid stream. One or more probe(s) and a detection molecule are selected. The probe(s) includes at least one lectin polypeptide probe configured to bind to a glycosylation site of one or more glycosylated protein(s). The glycosylated protein(s) are detected by quantifying the detection molecule.

In some embodiments, the one or more probes is at least one lectin polypeptide probe configured to bind a glycosylation site of the one or more glycosylated proteins. In some embodiments, the at least one lectin polypeptide probe has an amino acid sequence of SEQ ID NO. 1. In further embodiments, the at least one lectin polypeptide probe is an Aleuria aurantia lectin (AAL) probe or a Lens culinaris agglutinin (LCA) probe. In some embodiments, the AAL probe is conjugated to an R-Phycoerythrin protein. In some embodiments, the at least one lectin polypeptide probe is a microvesicle.

In some embodiments, the method may further include multiplexing the sample with a plurality of lectin polypeptide probes. In further embodiments, the one or more probes may have at least one antibody configured to bind the one or more glycosylated proteins. In some embodiments, the at least one antibody is de-glycosylated. In some embodiments, the at least one antibody is anti-human alpha-1-fetoprotein IgG1.

In some embodiments of the method described herein, the sample is selected from the group consisting of cells, microvesicles, blood, serum, urine, and a combination thereof.

In some embodiments of the method described herein, the detection molecule is selected from the group consisting of a capture antibody, a fluorophore and a combination thereof. In some embodiments, the fluorophore is an R-Phycoerythrin protein. In some embodiments, the capture antibody is a polyclonal chicken IgY antibody. In some embodiments, the capture antibody is conjugated to the fluorophore.

In some embodiments, the method may further include binding a capture bead to the one or more probe(s). In some embodiments, the capture bead has a size from about 5 microns to about 15 microns. In some embodiments, the one or more glycosylated proteins may include AFP, AFU, alpha-glucoside, bFGF, glypican-3, alpha-1-fucosidase, gamma-glutamyl transferase, golgi phosphoprotein 2, transforming growth factor beta, tumor specific growth factor, or hepatocyte growth factor.

In some embodiments of the method described herein, the glycosylation site is selected from the group consisting of alpha-1,6 fucosylation site, L-fucopyranosyl, alpha 1-2 L-fucopyranosyl, alpha 1-3 L-fucopyranosyl, and alpha 1-4 L-fucopyranosyl.

Also described herein is a nucleic acid having a nucleic acid sequence of SEQ ID NO. 2.

In some embodiments, the nucleic acid sequence of SEQ ID NO. 2 is:

ATGGACCGGCGGGTCGAGCCAGAATGTAATCGGCGAAGCAAAGCTTTTT
TCGCCACTGGCTGCTGTCACGTGGAAAAGTGCTCAGGGCATACAGATCC
GTGTTTACTGCGTCAATAAGGATAACATCCTCTCCGAATTTGTGTATGA
CGGTTCGAAGTGGATCACCGGAAACCTGGGCAGTGTCGGCGTCAAGGTG
GGCTCCAATTCGAAGCTTGCTGCGCTTCAGTGGGGCGGATCTGAGAGCG
CCCCCCCAAAAATCCGAGTTTACTACCAGAAGAGCAACGGTAGTGGGAG
CTCAATCCACGAGTATGTCTGGTCGGGCAAATGGACGGCTGGCGCAAGC
TTTGGGTCAACGGTGCCAGGAACGGGTATCGGAGCCACCGCCATCGGGC
CAGGTCGCCTGAGGATCTACTACCAGGCTACTGACAACAAGATCCGTGA
GCACTGTTGGGACTCCAACAGTTGGTACGTGGGGGGGTTCTCGGCCAGC
GCTTCCGCCGGCGTCTCCATCGCGGCGATTTCTTGGGGCAGTACACCCA
ACATCCGGGTCTACTGGCAGAAAGGTAGGGAGGAATTGTACGAGGCTGC
CTATGGCGGTTCATGGAACACTCCTGGTCAGATCAAGGACGCATCCAGG
CCTACGCCCTCGTTGCCAGACACCTTTATTGCTGCGAACTCCTCGGGGA
ACATCGACATCTCTGTGTTCTTCCAACTCGAGCATCATCATCACCATCA
CCACCATCATCAT.

Also described herein is a cDNA molecule encoding the nucleic acid sequence of SEQ ID NO. 2, along with an expression vector having the cDNA molecule. Further described herein is a nucleic acid having a nucleic acid sequence capable of transcribing the engineered lectin polypeptide.

Various aspects of the present disclosure may be further characterized by one or more of the following clauses:

Clause 1: An engineered lectin polypeptide comprising an amino acid sequence SEQ ID NO. 1.

Clause 2: The engineered lectin polypeptide of clause 1, wherein the lectin polypeptide binds to one or more glycosylation site(s) of a protein.

Clause 3: The engineered lectin polypeptide of clause 1 or 2, wherein the protein is selected from the group consisting of AFP, AFP-L3, AFU, alpha-glucoside, bFGF, glypican-3, alpha-1-fucosidase, gamma-glutamyl transferase, golgi phosphoprotein 2, transforming growth factor beta, tumor specific growth factor, and hepatocyte growth factor.

Clause 4: The engineered lectin polypeptide of clause 1 or 2, wherein the protein is AFP.

Clause 5: The engineered lectin polypeptide of clause 1 or 2, wherein the protein is AFP-L3.

Clause 6: The engineered lectin polypeptide of clause 1 or 2, wherein the protein is AFU.

Clause 7: The engineered lectin polypeptide of clause 1 or 2, wherein the protein is alpha-glucoside.

Clause 8: The engineered lectin polypeptide of clause 1 or 2, wherein the protein is bFGF.

Clause 9: The engineered lectin polypeptide of clause 1 or 2, wherein the protein is glypican-3.

Clause 10: The engineered lectin polypeptide of clause 1 or 2, wherein the protein is alpha-1-fucosidase.

Clause 11: The engineered lectin polypeptide of clause 1 or 2, wherein the protein is gamma-glutamyl transferase.

Clause 12: The engineered lectin polypeptide of clause 1 or 2, wherein the protein is golgi phosphoprotein 2.

Clause 13: The engineered lectin polypeptide of clause 1 or 2, wherein the protein is transforming growth factor beta.

Clause 14: The engineered lectin polypeptide of clause 1 or 2, wherein the protein is tumor specific growth factor.

Clause 15: The engineered lectin polypeptide of clause 1 or 2, wherein the protein is hepatocyte growth factor.

Clause 16: The engineered lectin polypeptide of any one of clauses 1 to 15, wherein the one or more glycosylation site(s) is alpha-1,6 fucosylation site, L-fucopyranosyl, alpha 1-2 L-fucopyranosyl, alpha 1-3 L-fucopyranosyl, or alpha 1-4 L-fucopyranosyl.

Clause 17: The engineered lectin polypeptide of any one of clauses 1 to 15, wherein the one or more glycosylation site(s) is alpha-1,6 fucosylation site.

Clause 18: The engineered lectin polypeptide of any one of clauses 1 to 15, wherein the one or more glycosylation site(s) is L-fucopyranosyl.

Clause 19: The engineered lectin polypeptide of any one of clauses 1 to 15, wherein the one or more glycosylation site(s) is alpha 1-2 L-fucopyranosyl.

Clause 20: The engineered lectin polypeptide of any one of clauses 1 to 15, wherein the one or more glycosylation site(s) is alpha 1-3 L-fucopyranosyl.

Clause 21: The engineered lectin polypeptide of any one of clauses 1 to 15, wherein the one or more glycosylation site(s) is alpha 1-4 L-fucopyranosyl.

Clause 22: The engineered lectin polypeptide of any one of clauses 1 to 21, further comprising a detection molecule.

Clause 23: The engineered lectin polypeptide of any one of clauses 1 to 22, wherein the detection molecule is selected from the group consisting of a capture antibody, a capture bead, a fluorophore and a combination thereof.

Clause 24: The engineered lectin polypeptide of any one of clauses 1 to 22, wherein the detection molecule is a capture antibody.

Clause 25: The engineered lectin polypeptide of any one of clauses 1 to 22, wherein the detection molecule is a capture bead.

Clause 26: The engineered lectin polypeptide of any one of clauses 1 to 22, wherein the detection molecule is a fluorophore.

Clause 27: The engineered lectin polypeptide of any one of clauses 1 to 22, wherein the detection molecule is a capture antibody and a capture bead.

Clause 28: The engineered lectin polypeptide of any one of clauses 1 to 22, wherein the detection molecule is a capture antibody and a fluorophore.

Clause 29: The engineered lectin polypeptide of any one of clauses 1 to 22, wherein the detection molecule is a capture bead and a fluorophore.

Clause 30: The engineered lectin polypeptide of any one of clauses 1 to 22, wherein the detection molecule is a capture antibody, a capture bead, and a fluorophore.

Clause 31: The engineered lectin polypeptide of any one of clauses 1 to 30, wherein the capture bead has a size from about 5 microns to about 15 microns.

Clause 32: The engineered lectin polypeptide of any one of clauses 1 to 30, wherein the capture bead has a size from about 6 microns to about 14 microns.

Clause 33: The engineered lectin polypeptide of any one of clauses 1 to 30, wherein the capture bead has a size from about 7 microns to about 13 microns.

Clause 34: The engineered lectin polypeptide of any one of clauses 1 to 30, wherein the capture bead has a size from about 8 microns to about 12 microns.

Clause 35: The engineered lectin polypeptide of any one of clauses 1 to 30, wherein the capture bead has a size from about 9 microns to about 11 microns.

Clause 36: The engineered lectin polypeptide of any one of clauses 1 to 30, wherein the capture bead has a size of about 5 microns.

Clause 37: The engineered lectin polypeptide of any one of clauses 1 to 30, wherein the capture bead has a size of about 6 microns.

Clause 38: The engineered lectin polypeptide of any one of clauses 1 to 30, wherein the capture bead has a size of about 7 microns.

Clause 39: The engineered lectin polypeptide of any one of clauses 1 to 30, wherein the capture bead has a size of about 8 microns.

Clause 40: The engineered lectin polypeptide of any one of clauses 1 to 30, wherein the capture bead has a size of about 9 microns.

Clause 41: The engineered lectin polypeptide of any one of clauses 1 to 30, wherein the capture bead has a size of about 10 microns.

Clause 42: The engineered lectin polypeptide of any one of clauses 1 to 30, wherein the capture bead has a size of about 11 microns.

Clause 43: The engineered lectin polypeptide of any one of clauses 1 to 30, wherein the capture bead has a size of about 12 microns.

Clause 44: The engineered lectin polypeptide of any one of clauses 1 to 30, wherein the capture bead has a size of about 13 microns.

Clause 45: The engineered lectin polypeptide of any one of clauses 1 to 30, wherein the capture bead has a size of about 14 microns.

Clause 46: The engineered lectin polypeptide of any one of clauses 1 to 30, wherein the capture bead has a size of about 15 microns.

Clause 47: The engineered lectin polypeptide of any one of clauses 1 to 46, wherein the engineered lectin polypeptide is an Aleuria aurantia lectin (AAL) or a Lens culinaris agglutinin (LCA) probe.

Clause 48: The engineered lectin polypeptide of any one of clauses 1 to 46, wherein the engineered lectin polypeptide is an Aleuria aurantia lectin (AAL) probe.

Clause 49: The engineered lectin polypeptide of any one of clauses 1 to 46, wherein the engineered lectin polypeptide is a Lens culinaris agglutinin (LCA) probe.

Clause 50: The engineered lectin polypeptide of any one of clauses 1 to 49, wherein the Aleuria aurantia lectin has three or more fucosylated oligosaccharide binding sites.

Clause 51: A method of flow cytometry for detecting a glycosylation site in a sample comprising establishing a fluid stream, adding a sample having one or more glycosylated protein(s) to the fluid stream, selecting one or more probe(s) and a detection molecule, and detecting the one or more glycosylated protein(s) by quantifying the detection molecule, wherein the one or more probe(s) comprises at least one lectin polypeptide probe configured to bind a glycosylation site of the one or more glycosylated protein(s).

Clause 52: The method of clause 51, wherein the at least one lectin polypeptide probe comprises amino acid sequence SEQ ID NO. 1.

Clause 53: The method of clause 51 or 52, wherein the at least one lectin polypeptide probe is an Aleuria aurantia lectin (AAL) or a Lens culinaris agglutinin (LCA) probe.

Clause 54: The method of clause 51 or 52, wherein the at least one lectin polypeptide probe is an Aleuria aurantia lectin (AAL) probe.

Clause 55: The method of clause 51 or 52, wherein the at least one lectin polypeptide probe is a Lens culinaris agglutinin (LCA) probe.

Clause 56: The method of any one of clauses 51 to 55, wherein the AAL vector is conjugated to an R-Phycoerythrin protein.

Clause 57: The method of any one of clauses 51 to 56, wherein the at least one lectin polypeptide probe comprises a microvesicle.

Clause 58: The method of any one of clauses 51 to 57, further comprising multiplexing the sample with a plurality of lectin polypeptide probes.

Clause 59: The method of any one of clauses 51 to 58, wherein the one or more probe(s) comprises at least one antibody configured to bind the one or more glycosylated protein(s).

Clause 60: The method of any one of clauses 51 to 59, wherein the at least one antibody is de-glycosylated.

Clause 61: The method of any one of clauses 51 to 60, wherein the at least one antibody is anti-human alpha-1 fetoprotein IgG1.

Clause 62: The method of any one of clauses 51 to 61, wherein the sample is selected from the group consisting of cells, microvesicles, blood, serum, urine, and a combination thereof.

Clause 63: The method of any one of clauses 51 to 61, wherein the sample is cells.

Clause 64: The method of any one of clauses 51 to 61, wherein the sample is microvesicles.

Clause 65: The method of any one of clauses 51 to 61, wherein the sample is blood.

Clause 66: The method of any one of clauses 51 to 61, wherein the sample is serum.

Clause 67: The method of any one of clauses 51 to 61, wherein the sample is urine.

Clause 68: The method of any one of clauses 51 to 61, wherein the sample is cells and microvesicles.

Clause 69: The method of any one of clauses 51 to 61, wherein the sample is cells and blood.

Clause 70: The method of any one of clauses 51 to 61, wherein the sample is cells and serum.

Clause 71: The method of any one of clauses 51 to 61, wherein the sample is cells and urine.

Clause 72: The method of any one of clauses 51 to 61, wherein the sample is microvesicles and blood.

Clause 73: The method of any one of clauses 51 to 61, wherein the sample is microvesicles and serum.

Clause 74: The method of any one of clauses 51 to 61, wherein the sample is microvesicles and urine.

Clause 75: The method of any one of clauses 51 to 61, wherein the sample is blood and serum.

Clause 76: The method of any one of clauses 51 to 61, wherein the sample is blood and urine.

Clause 77: The method of any one of clauses 51 to 61, wherein the sample is serum and urine.

Clause 78: The method of any one of clauses 51 to 61, wherein the sample is cells, microvesicles, and blood.

Clause 79: The method of any one of clauses 51 to 61, wherein the sample is cells, microvesicles, and serum.

Clause 80: The method of any one of clauses 51 to 61, wherein the sample is cells, microvesicles, and urine.

Clause 81: The method of any one of clauses 51 to 61, wherein the sample is cells, blood, and serum.

Clause 82: The method of any one of clauses 51 to 61, wherein the sample is cells, blood, and urine.

Clause 83: The method of any one of clauses 51 to 61, wherein the sample is cells, serum, and blood.

Clause 84: The method of any one of clauses 51 to 61, wherein the sample is cells, serum, and urine.

Clause 85: The method of any one of clauses 51 to 61, wherein the sample is cells, urine, and blood.

Clause 86: The method of any one of clauses 51 to 61, wherein the sample is cells, urine, and serum.

Clause 87: The method of any one of clauses 51 to 61, wherein the sample is cells, microvesicles, blood, and serum.

Clause 88: The method of any one of clauses 51 to 61, wherein the sample is cells, microvesicles, blood, and urine.

Clause 89: The method of any one of clauses 51 to 61, wherein the sample is cells, microvesicles, blood, serum, and urine.

Clause 90: The method of any one of clauses 51 to 89, wherein the detection molecule is selected from the group consisting of a capture antibody, a fluorophore and a combination thereof.

Clause 91: The method of any one of claims 51 to 89, wherein the detection molecule is a capture antibody.

Clause 92: The method of any one of claims 51 to 89, wherein the detection molecule is a fluorophore.

Clause 93: The method of any one of claims 51 to 89, wherein the detection molecule is a capture antibody and a fluorophore.

Clause 94: The method of any one of clauses 51 to 93, wherein the fluorophore is an R-Phycoerythrin protein.

Clause 95: The method of any one of clauses 51 to 93, wherein the capture antibody is a polyclonal chicken IgY antibody.

Clause 96: The method of any one of clauses 51 to 95, wherein the capture antibody is conjugated to the fluorophore.

Clause 97: The method of any one of clauses 51 to 96, further comprising binding a capture bead to the one or more probe(s).

Clause 98: The method of any one of clauses 51 to 97, wherein the capture bead has a size from about 5 microns to about 15 microns.

Clause 99: The method of any one of clauses 51 to 97: wherein the capture bead has a size from about 6 microns to about 14 microns.

Clause 100: The method of any one of clauses 51 to 97: wherein the capture bead has a size from about 7 microns to about 13 microns.

Clause 101: The method of any one of clauses 51 to 97: wherein the capture bead has a size from about 8 microns to about 12 microns.

Clause 102: The method of any one of clauses 51 to 97: wherein the capture bead has a size from about 9 microns to about 11 microns.

Clause 103: The method of any one of clauses 51 to 97: wherein the capture bead has a size of about 5 microns.

Clause 104: The method of any one of clauses 51 to 97: wherein the capture bead has a size of about 6 microns.

Clause 105: The method of any one of clauses 51 to 97: wherein the capture bead has a size of about 7 microns.

Clause 106: The method of any one of clauses 51 to 97: wherein the capture bead has a size of about 8 microns.

Clause 108: The method of any one of clauses 51 to 97: wherein the capture bead has a size of about 9 microns.

Clause 109: The method of any one of clauses 51 to 97: wherein the capture bead has a size of about 10 microns.

Clause 110: The method of any one of clauses 51 to 97: wherein the capture bead has a size of about 11 microns.

Clause 111: The method of any one of clauses 51 to 97: wherein the capture bead has a size of about 12 microns.

Clause 112: The method of any one of clauses 51 to 97: wherein the capture bead has a size of about 13 microns.

Clause 113: The method of any one of clauses 51 to 97: wherein the capture bead has a size of about 14 microns.

Clause 114: The method of any one of clauses 51 to 97: wherein the capture bead has a size of about 15 microns.

Clause 115: The method of any one of clauses 51 to 114, wherein the one or more glycosylated proteins comprises AFP, AFP-L3, AFU, alpha-glucoside, bFGF, glypican-3, alpha-1-fucosidase, gamma-glutamyl transferase, golgi phosphoprotein 2, transforming growth factor beta, tumor specific growth factor, or hepatocyte growth factor.

Clause 116: The method of any one of clauses 51 to 114: wherein the one or more glycosylated proteins is AFP.

Clause 117: The method of any one of clauses 51 to 114: wherein the one or more glycosylated proteins is AFP-L3.

Clause 118: The method of any one of clauses 51 to 114: wherein the one or more glycosylated proteins is AFU.

Clause 119: The method of any one of clauses 51 to 114: wherein the one or more glycosylated proteins is alpha-glucoside.

Clause 120: The method of any one of clauses 51 to 114: wherein the one or more glycosylated proteins is bFGF.

Clause 121: The method of any one of clauses 51 to 114: wherein the one or more glycosylated proteins is glypican-3.

Clause 122: The method of any one of clauses 51 to 114: wherein the one or more glycosylated proteins is alpha-1-fucosidase.

Clause 123: The method of any one of clauses 51 to 114: wherein the one or more glycosylated proteins is gamma-glutamyl transferase.

Clause 124: The method of any one of clauses 51 to 114: wherein the one or more glycosylated proteins is golgi phosphoprotein 2.

Clause 125: The method of any one of clauses 51 to 114: wherein the one or more glycosylated proteins is transforming growth factor beta.

Clause 126: The method of any one of clauses 51 to 114: wherein the one or more glycosylated proteins is tumor specific growth factor.

Clause 127: The method of any one of clauses 51 to 114: wherein the one or more glycosylated proteins is hepatocyte growth factor.

Clause 128: The method of any one of clauses 51 to 127, wherein the glycosylation site is selected from the group consisting of alpha-1,6 fucosylation site, L-fucopyranosyl, alpha 1-2 L-fucopyranosyl, alpha 1-3 L-fucopyranosyl, and alpha 1-4 L-fucopyranosyl.

Clause 129: The method of any one of clauses 51 to 127, wherein the glycosylation site is alpha-1,6 fucosylation site.

Clause 130: The method of any one of clauses 51 to 127, wherein the glycosylation site is L-fucopyranosyl.

Clause 131: The method of any one of clauses 51 to 127, wherein the glycosylation site is alpha 1-2 L-fucopyranosyl.

Clause 132: The method of any one of clauses 51 to 127, wherein the glycosylation site is alpha 1-3 L-fucopyranosyl.

Clause 133: The method of any one of clauses 51 to 127, wherein the glycosylation site is alpha 1-4 L-fucopyranosyl.

Clause 134: A nucleic acid comprising a nucleic acid sequence SEQ ID NO. 2.

Clause 135: A cDNA molecule encoding the nucleic acid of clause 134.

Clause 136: An expression vector comprising the cDNA of clause 134 or 135.

Clause 137: A nucleic acid comprising a nucleic acid sequence capable of transcribing the engineered lectin polypeptide of any one of clauses 1 to 50.

### BRIEF DESCRIPTION OF THE DRAWING(S)

FIG. 1 is an illustration of an embodiment of the method of flow cytometry for detecting a glycosylation site in a sample.

FIG. 2 is a depiction of a glycoflow based method for early detection of Hepatocellular Carcinoma (HCC).

FIG. 3 is an illustration of titration of AFP into a glycoflow assay with a capture bead.

FIG. 4 depicts glycan analysis of 5 μg AFP from a healthy patient.

FIG. 5 is a depiction of a glycoflow based method for early detection of Hepatocellular Carcinoma (HCC) wherein the capture bead is coated with streptavidin and the capture antibody is coated or tagged with biotin.

### DESCRIPTION OF THE INVENTION

For purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise. For example, “a” diluent, “an” intragranular excipient, “a” disintegrant, and the like refer to one or more of any of these items.

“About” as used herein means±10% of the referenced value. In certain embodiments, “about” means±9%, or ±8%, or ±7%, or ±6%, or ±5%, or ±4%, or ±3%, or ±2% or ±1% of the referenced value.

In one aspect, the present disclosure is directed to an engineered lectin polypeptide having an amino acid sequence of SEQ ID NO. 1, wherein SEQ ID NO. 1 has the following amino acid sequence:

MPTEFLYTSKIAAISWAATGGRQQRVYFQDLNGKIREAQRG
GDNPWTGGSSQNVIGEAKLFSPLAAVTWKSAQGIQIRVYCV
NKDNILSEFVYDGSKWITGNLGSVGVKVGSNSKLAALQWGG
SESAPPNIRVYYQKSNGSGSSIHEYVWSGKWTAGASFGSTVP
GTGIGATAIGPGRLRIYYQATDNKIREHCWDSNSWYVGGFSA
SASAGVSIAAISWGSTPNIRVYWQKGREELYEAAYGGSWNT
PGQIKDASRPTPSLPDTFIAANSSGNIDISVFFQASGVSLQQW
QWISGKGWSIGAVVPTGTPAGWLEHHHHHHHHHH.

In some embodiments, the amino acid sequence of SEQ ID NO. 1 is a mutant amino acid sequence wherein amino acid 101 of the sequence has been mutated from a glutamine (Q) amino acid to an asparagine (N) amino acid.

In some embodiments, the amino acid sequence of SEQ ID NO. 1 binds to one or more glycosylation site(s) on a protein. A glycosylation site on a protein is understood as a site on the protein wherein a carbohydrate (i.e., a glycosyl donor) is attached to a hydroxyl or other functional group of the protein. Further, glycosylation may refer to an enzymatic process that attaches glycans to a glycosylation site on the protein. Glycosylation can be a form of co-translational or post-translational modification. In some embodiments of the present disclosure, the glycosylation site(s) on the protein can be one or more of the following: an alpha-1,6 fucosylation site, a L-fucopyranosyl, an alpha 1-2 L-fucopyranosyl site, an alpha 1-3 L-fucopyranosyl site, or an alpha 1-4 L-fucopyranosyl.

In some embodiments, the protein is any protein that is capable of being glycosylated. The protein, and the protein's glycosylated isoforms, may serve as a biomarker for a particular disease. In some embodiments, the protein can be AFP, AFP-L3, AFU, alpha-glucoside, bFGF, glypican-3, alpha-1-fucosidase, gamma-glutamyl transferase, golgi phosphoprotein 2, transforming growth factor beta, tumor specific growth factor, or hepatocyte growth factor.

The engineered lectin polypeptide may also have a detection molecule. The detection molecule can generally be any molecule capable of binding to the engineered lectin polypeptide or the protein having one or more glycosylation sites. In some embodiments, the detection molecule can be a capture antibody, a capture bead, a fluorophore or a combination thereof. In some embodiments, the detection molecule is a capture bead having a size from about 5 microns to about 15 microns, from about 6 microns to about 14 microns, from about 7 microns to about 13 microns, from about 8 microns to about 12 microns, or from about 9 microns to about 11 microns. In some embodiments, the detection molecule is a capture bead having a size of about 5 microns, 6 microns, 7 microns, 8 microns, 9 microns, 10 microns, 11 microns, 12 microns, 13 microns, 14 microns, or 15 microns.

In some embodiments, the detection molecule is a capture bead that is coated with streptavidin. In some embodiments, the detection molecule is a capture antibody that is coated or tagged with biotin. As will be understood by those skilled in the art biotinylation is a chemical or enzymatic process which incorporates biotin onto a protein or antigen. Chemical biotinylation utilizes various conjugation chemistries to yield specific or nonspecific biotinylation of amines, carboxylates, sulfhydryl's and carbohydrates. Enzymatic biotinylation provides biotinylation of a specific lysine within a certain sequence of a protein or antigen by a biotin ligase. Biotin binds to streptavidin and avidin with high affinity, a fast on-rate, and high specificity, and these interactions are exploited in many areas of biotechnology to isolate biotinylated molecules of interest.

In some embodiments, the engineered lectin polypeptide is an Aleuria aurantia lectin (AAL) probe or a Lens culinaris agglutinin (LCA) probe. An LCA vector is a type of vector that recognizes proteins or amino acids containing α-linked mannose residues or additional sugars.

In embodiments where the engineered lectin polypeptide is an Aleuria aurantia lectin probe, the Aleuria aurantia lectin probe may have three or more fucosylated oligosaccharide binding sites. AAL is a 312 amino acid protein which contains five binding sites for L-fucose or L-fucose-linked oligosaccharides. The multivalent nature of AAL gives it an unusually high binding affinity (micromolar) for fucosylated carbohydrate ligands compared to other lectins.

Commercial production of AAL isolates and purifies the lectin by binding to a fucose-starch column. AAL is eluted from the column with 50 mM L-fucose (Fujihashi et al.). Structural and biochemical analysis has shown that commercial AAL has 3 to 5 of its 5 ligand binding sites occupied with fucose as a result of this manufacturing process (Olausson et al., Amano et al., Fujihashi et al., and Wimmerova et al.).

In some embodiments, recombinant wild-type AAL as well as recombinantly engineered forms of AAL are contemplated herein. Methods are provided that include the creation and production of mutant AAL proteins altered either by site directed mutagenesis or by random mutagenesis and subsequently selected for high binding affinity for glycosylation sites contemplated herein. Preferably, and as contemplated herein, the engineered lectin polypeptide is an AAL probe having amino acid sequence SEQ ID NO. 1 and has high binding affinity for L-fucopyranosyl or alpha-1,6 fucosylation site(s). Further, the AAL probes contemplated herein have a high affinity for the outer arm L-fucopyranosyl linkages, more specifically mutated AAL protein having high affinity for the alpha 1-2 outer arm L-fucopyranosyl linkage, alpha 1-3 outer arm L-fucopyranosyl linkage, or alpha 1-4 outer arm L-fucopyranosyl linkage and core fucosylated alpha-1,6 fucosylation linkage found in serum protein biomarkers in patients with diseases such as, but not limited to, cancer.

Surprisingly, it was found that recombinant AAL produced in and isolated from bacteria using nickel affinity chromatography had substantially higher binding affinities for fucosylated oligosaccharides than commercially prepared AAL as determined by surface plasmon resonance studies, tryptophan fluorescence studies and enzyme linked lectin assays.

In some embodiments, recombinant AAL is incorporated as a probe or detector molecule in the flow cytometry platform described herein.

The present disclosure is further directed towards the use of an engineered lectin polypeptide in a flow cytometry platform to measure alterations in glycosylation sites on proteins for the detection of disease such as cancer (e.g., HCC). In some embodiments of the present disclosure, a flow cytometry platform is used to detect glycosylated isoforms in a patient sample for individuals with inflammatory disorders, autoimmune disorders, cancer, infections, or other disorders where a change in the glycosylation sites of specific proteins are used as biomarkers in serum or as biomarkers expressed on the surface of cells, or microvesicles derived from cells. Analysis of the levels of these proteins, either through identification of the glycosylated isoform or quantification of the protein levels expressing these glycosylated isoforms, provides for a flow cytometry based platform for detecting patients with disease or people at risk for disease progression. The flow cytometry method incorporating the use of an engineered lectin polypeptide as a probe in the flow cytometry platform is further contemplated below.

In one aspect, the present disclosure is directed to a method of flow cytometry for detecting a glycosylation site in a sample. The method includes establishing a fluid stream. A sample having one or more glycosylated protein(s) is added to the fluid stream. One or more probe(s) and a detection molecule are selected. The probe(s) includes at least one lectin polypeptide probe configured to bind to a glycosylation site of one or more glycosylated protein(s). The glycosylated protein(s) are detected by quantifying the detection molecule.

In some embodiments, the at least one lectin polypeptide probe has an amino acid sequence of SEQ ID NO. 1, wherein SEQ ID NO. 1 has the following amino acid sequence:

MPTEFLYTSKIAAISWAATGGRQQRVYFQDLNGKIREAQRG
GDNPWTGGSSQNVIGEAKLFSPLAAVTWKSAQGIQIRVYCV
NKDNILSEFVYDGSKWITGNLGSVGVKVGSNSKLAALQWGG
SESAPPNIRVYYQKSNGSGSSIHEYVWSGKWTAGASFGSTVP
GTGIGATAIGPGRLRIYYQATDNKIREHCWDSNSWYVGGFSA
SASAGVSIAAISWGSTPNIRVYWQKGREELYEAAYGGSWNT
PGQIKDASRPTPSLPDTFIAANSSGNIDISVFFQASGVSLQQW
QWISGKGWSIGAVVPTGTPAGWLEHHHHHHHHHH.

In some embodiments, the amino acid sequence of SEQ ID NO. 1 is a mutant amino acid sequence wherein amino acid 101 of the sequence has been mutated from a glutamine (Q) amino acid to an asparagine (N) amino acid.

In some embodiments, the at least one lectin polypeptide probe is an AAL probe or a LCA probe. In further embodiments, the AAL or LCA probe may have the amino acid sequence of SEQ ID NO. 1 that has an asparagine (N) amino acid at amino acid site 101 in place of a glutamine (Q) amino acid. In some embodiments, the AAL vector having SEQ ID NO. 1 is conjugated to a fluorescence-based indicator such as, but not limited to, R-Phycoerythrin protein.

In further embodiments, the at least one lectin polypeptide probe may have a microvesicle. In further embodiments, the microvesicle may have the amino acid sequence of SEQ ID NO 1. In some embodiments, the microvesicle having SEQ ID NO. 1 is conjugated to a fluorescence-based indicator.

In another aspect of the method, the one or more probe(s) may have at least one antibody configured to bind the one or more glycosylated protein(s). The one or more glycosylated protein(s) may include AFP, AFP-L3, AFU, alpha-glucoside, bFGF, glypican-3, alpha-1-fucosidase, gamma-glutamyl transferase, golgi phosphoprotein 2, transforming growth factor beta, tumor specific growth factor, or hepatocyte growth factor. In some embodiments, the at least one antibody is de-glycosylated. In further embodiments, the at least one antibody is de-glycosylated before binding to the one or more glycosylated protein(s). In a preferred embodiment, the at least one antibody is anti-human alpha-1 fetoprotein IgG1.

In some embodiments, the glycosylation site detected in the sample can be one or more of the following: an alpha-1,6 fucosylation site, a L-fucopyranosyl, an alpha 1-2 L-fucopyranosyl site, an alpha 1-3 L-fucopyranosyl site, or an alpha 1-4 L-fucopyranosyl.

In some embodiments of the method contemplated herein, the sample for detecting a glycosylation site is from a subject, mammal, or patient, preferably from a human patient. The sample may include cells, microvesicle, blood, serum, urine, or a combination thereof from the patient.

In further aspects of the method, the detection molecule can include any molecule capable of binding to the at least one lectin polypeptide probe or the one or more glycosylated protein(s). In some embodiments, the detection molecule can be a capture antibody, a fluorophore, or a combination thereof. In some embodiments, the fluorophore is an R-Phycoerythrin protein. In further embodiments, the capture antibody is a polyclonal chicken IgY antibody. In some embodiments, the capture antibody is conjugated to the fluorophore. In some embodiments, the capture antibody is coated or tagged with biotin.

In further aspects of the method disclosed herein, a capture bead is bound to the one or more probes. In some embodiments, the capture bead has a size from about 5 microns to about 15 microns, from about 6 microns to about 14 microns, from about 7 microns to about 13 microns, from about 8 microns to about 12 microns, or from about 9 microns to about 11 microns. In some embodiments, the detection molecule is a capture bead having a size of about 5 microns, 6 microns, 7 microns, 8 microns, 9 microns, 10 microns, 11 microns, 12 microns, 13 microns, 14 microns, or 15 microns. In further embodiments, the capture bead is coated with streptavidin.

The method of flow cytometry for detecting a glycosylation site may further include multiplexing the sample with a plurality of lectin polypeptide probes in the sample. As used herein, multiplexing is a type of assay or method used in the flow cytometry platform contemplated herein for detecting one or more glycosylation site(s) of one or more glycosylated protein(s) in a sample with a plurality of lectin polypeptide probes. The plurality of lectin polypeptide probes may be configured to bind one or more glycosylation site(s) of the one or more glycosylated protein(s). The plurality of lectin polypeptide probes may include amino acid sequence of SEQ ID NO. 1. The plurality of polypeptide probes may further include an AAL probe or an LCA probe as described herein. The plurality of polypeptide probes may further include at least one antibody configured to bind the one or more glycosylated protein(s).

In some embodiments, the one or more glycosylated protein(s) may include AFP, AFP-L3, AFU, alpha-glucoside, bFGF, glypican-3, alpha-1-fucosidase, gamma-glutamyl transferase, golgi phosphoprotein 2, transforming growth factor beta, tumor specific growth factor, or hepatocyte growth factor. In further embodiments, the one or more glycosylation site(s) may include alpha-1,6 fucosylation site, L-fucopyranosyl, alpha 1-2 L-fucopyranosyl, alpha 1-3 L-fucopyranosyl, and alpha 1-4 L-fucopyranosyl.

In a further aspect, the present disclosure is directed to a nucleic acid having a nucleic acid sequence of SEQ ID NO. 2, wherein SEQ ID NO. 2 has the following nucleic acid sequence:

ATGGACCGGCGGGTCGAGCCAGAATGTAATCGGCGAAGCAAAGCTTTTT
TCGCCACTGGCTGCTGTCACGTGGAAAAGTGCTCAGGGCATACAGATCC
GTGTTTACTGCGTCAATAAGGATAACATCCTCTCCGAATTTGTGTATGA
CGGTTCGAAGTGGATCACCGGAAACCTGGGCAGTGTCGGCGTCAAGGTG
GGCTCCAATTCGAAGCTTGCTGCGCTTCAGTGGGGCGGATCTGAGAGCG
CCCCCCCAAAAATCCGAGTTTACTACCAGAAGAGCAACGGTAGTGGGAG
CTCAATCCACGAGTATGTCTGGTCGGGCAAATGGACGGCTGGCGCAAGC
TTTGGGTCAACGGTGCCAGGAACGGGTATCGGAGCCACCGCCATCGGGC
CAGGTCGCCTGAGGATCTACTACCAGGCTACTGACAACAAGATCCGTGA
GCACTGTTGGGACTCCAACAGTTGGTACGTGGGGGGGTTCTCGGCCAGC
GCTTCCGCCGGCGTCTCCATCGCGGCGATTTCTTGGGGCAGTACACCCA
ACATCCGGGTCTACTGGCAGAAAGGTAGGGAGGAATTGTACGAGGCTGC
CTATGGCGGTTCATGGAACACTCCTGGTCAGATCAAGGACGCATCCAGG
CCTACGCCCTCGTTGCCAGACACCTTTATTGCTGCGAACTCCTCGGGGA
ACATCGACATCTCTGTGTTCTTCCAACTCGAGCATCATCATCACCATCA
CCACCATCATCAT.

In some embodiments, the nucleic acid sequence of SEQ ID NO. 2 is a mutant amino acid sequence wherein nucleic acids 170-172 of the sequence have been mutated to encode for an asparagine (N) amino acid. In some embodiments, SEQ ID NO. 2 is a messenger RNA sequence that encodes for the amino acid sequence of SEQ ID NO. 1. In further embodiments, a cDNA molecule is included that encodes for the nucleic acid sequence of SEQ ID NO. 2. In some embodiments, an expression vector includes the nucleic acid sequence of SEQ ID NO. 2 or the cDNA molecule that encodes for the nucleic acid sequence of SEQ ID NO. 2. In further embodiments, an AAL or LCA probe is within an expression vector and includes the nucleic acid sequence of SEQ ID NO. 2 or the cDNA molecule that encodes for the nucleic acid sequence of SEQ ID NO. 2.

In further embodiments, a nucleic acid having a nucleic acid sequence capable of transcribing the engineered lectin polypeptide is contemplated. In some embodiments, the nucleic acid sequence is SEQ ID NO. 2. In further embodiments, the nucleic acid sequence of SEQ ID NO. 2 is transcribed to SEQ ID NO. 1.

The following examples are presented to demonstrate the general principles of the invention of this disclosure. The invention should not be considered as limited to the specific example presented.

### Example 1

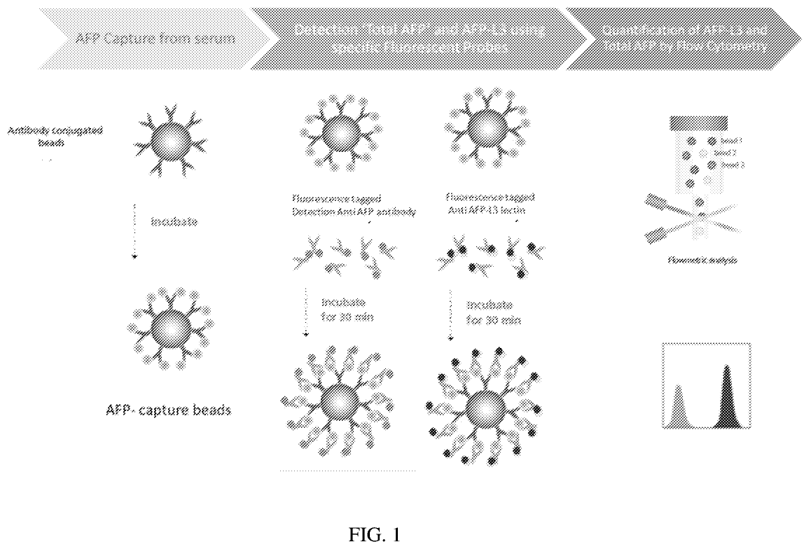

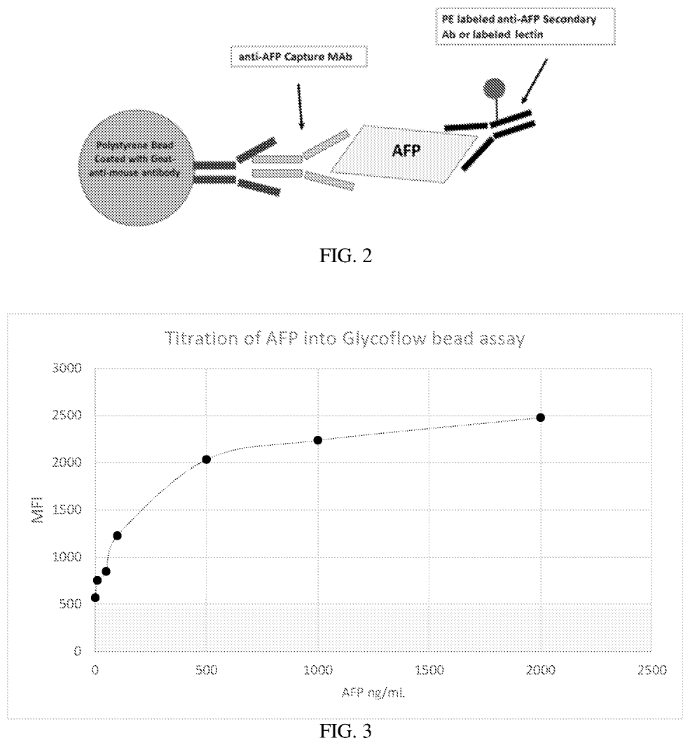

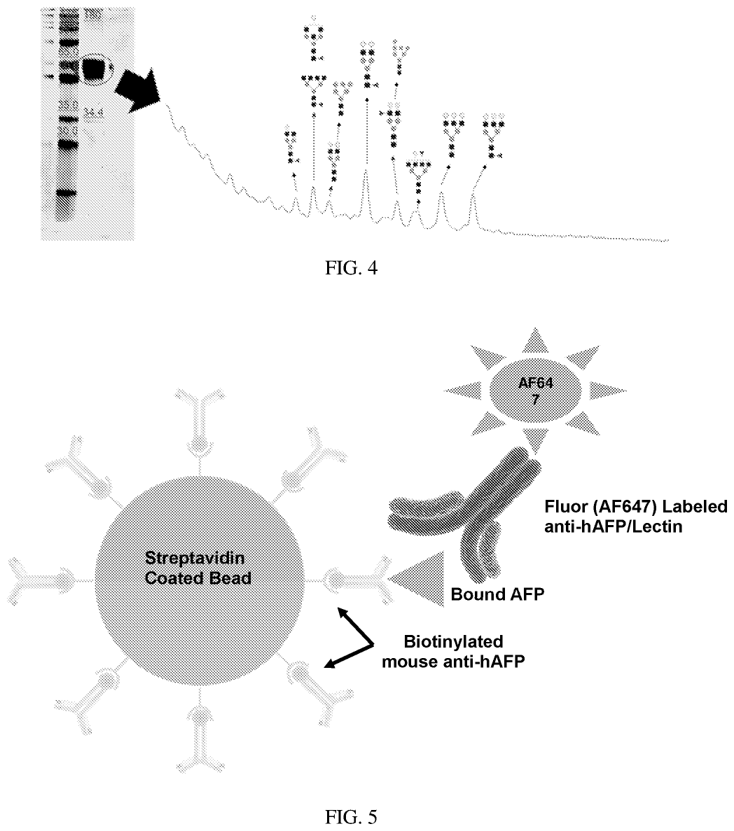

As depicted in FIGS. 1 and 2, total AFP is bound to capture beads coated with a mono or polyclonal anti-AFP antibody. This total AFP is then measured by the binding of fluorescently-tagged anti-AFP antibody targeting an exposed epitope of AFP. A glycosylated isoform of AFP (e.g., AFP-L3) may then be identified or quantified using a genetically modified AAL or LCA that has been fluorescently tagged with one or more fluorophores. This may allow for total AFP and the glycosylated isoform (e.g., AFP-L3) to be readily detected and quantified from serum using the flow cytometry platform disclosed herein. The flow cytometry platform disclosed herein may allow for the quantification of both AFP and AFP-L3 which could represent a powerful diagnostic tool for early detection of disease (e.g., HCC). The flow cytometry platform disclosed herein has advantages over the current FDA-approved immunofluorescent liquid phase binding assay in that the flow cytometry platform disclosed herein allows for increased sensitivity and accuracy (signal to noise ratio), diagnostic clarity, and multiplexing capabilities. As illustrated in FIGS. 3 and 4, the flow cytometry platform disclosed herein has the requisite selectivity and sensitivity for clinical diagnostic assays which utilize a flow cytometry platform.

### Example 2

Increased serum levels of fucosylated glycosylated isoforms of proteins may serve as early biomarkers of diseases such as cancer. Further, increased serum levels for a galactosylated glycosylated isoforms of immunoglobulin G, called alpha-gal IgG, may correlate with the diagnosis of liver disease. As such, recombinant or mutant forms of lectin (e.g., AAL or LCA), linked to a reporter molecule (e.g., a radiolabel, chromophore or fluorophore), can be used as an alpha-gal IgG detection molecule, specific for fucosylated proteins in blood. Incorporation into a bead-based assay system provides the basis for a flow cytometry method to determine a patient's disease status.

### Example 3

Simultaneous flow cytometric assays may be performed, for example, determining levels of AFP-L3 and another target protein biomarker (including Glypican-3, Alpha-1-fucosidase, Gamma-Glutamyl transferase, Golgi phosphoprotein 2, Transforming Growth Factor Beta, Tumor Specific Growth Factor, Hepatocyte Growth Factor, Basic Fibroblast Growth Factor) with internal determination of sample related non-specific binding (NSB). The assay may utilize capture beads of various sizes (typically 7.5 and 5.5 μm diameter) coated with monoclonal antibodies specific for AFP, or other target proteins disclosed herein, to capture these two targets from the serum samples. Secondary detection probes (engineered lectin in the case of AFP-L3 and monoclonal antibody in the case of second target protein biomarker) that have been tagged with different fluorophores can be used to label the captured AFP-L3 and the other target protein biomarker. The various capture beads can be identified by light-scatter measurements and by the different detection probes used to label each of the biomarkers. Each capture bead detected by flow cytometry will have a fluorescence signal that is proportional to the amount of AFP-L3 or other target protein biomarker bound. Using this method, it is possible to quantify biomarkers from serum samples, and eliminates many issues associated with serum testing by other methods such as high background and low detection.

1. An engineered lectin polypeptide comprising an amino acid sequence SEQ ID NO. 1.

2. The engineered lectin polypeptide of claim 1, wherein the lectin polypeptide binds to one or more glycosylation site(s) of a protein.

3. The engineered lectin polypeptide of claim 2, wherein the protein is selected from the group consisting of AFP, AFP-L3, AFU, alpha-glucoside, bFGF, glypican-3, alpha-1-fucosidase, gamma-glutamyl transferase, golgi phosphoprotein 2, transforming growth factor beta, tumor specific growth factor, and hepatocyte growth factor.

4. The engineered lectin polypeptide of claim 2, wherein the one or more glycosylation site(s) is alpha-1,6 fucosylation site, L-fucopyranosyl, alpha 1-2 L-fucopyranosyl, alpha 1-3 L-fucopyranosyl, or alpha 1-4 L-fucopyranosyl.

5. The engineered lectin polypeptide of claim 1, further comprising a detection molecule.

6. The engineered lectin polypeptide of claim 5, wherein the detection molecule is selected from the group consisting of a capture antibody, a capture bead, a fluorophore and a combination thereof.

7. The engineered lectin polypeptide of claim 6, wherein the capture bead has a size from about 5 microns to about 15 microns.

8. The engineered lectin polypeptide of claim 6, wherein the capture bead is coated with streptavidin.

9. The engineered lectin polypeptide of claim 6, wherein the capture antibody is coated or tagged with biotin.

10. The engineered lectin polypeptide of claim 1, wherein the engineered lectin polypeptide is an Aleuria aurantia lectin (AAL) or a Lens culinaris agglutinin (LCA) probe.

11. The engineered lectin polypeptide of claim 10, wherein the Aleuria aurantia lectin has three or more fucosylated oligosaccharide binding sites.

12. A method of flow cytometry for detecting a glycosylation site in a sample comprising:
establishing a fluid stream;
adding a sample having one or more glycosylated protein(s) to the fluid stream;
selecting one or more probe(s) and a detection molecule; and
detecting the one or more glycosylated protein(s) by quantifying the detection molecule,
wherein the one or more probe(s) comprises at least one lectin polypeptide probe configured to bind a glycosylation site of the one or more glycosylated protein(s).

13. The method of claim 12, wherein the at least one lectin polypeptide probe comprises amino acid sequence SEQ ID NO. 1.

14. The method of claim 12, wherein the at least one lectin polypeptide probe is an Aleuria aurantia lectin (AAL) probe or a Lens culinaris agglutinin (LCA) probe.

15. (canceled)

16. (canceled)

17. The method of claim 12, further comprising multiplexing the sample with a plurality of lectin polypeptide probes.

18. The method of claim 12, wherein the one or more probe(s) comprises at least one antibody configured to bind the one or more glycosylated protein(s).

19-21. (canceled)

22. The method of claim 12, wherein the detection molecule is selected from the group consisting of a capture antibody, a fluorophore and a combination thereof.

23-26. (canceled)

27. The method of claim 12, further comprising binding a capture bead to the one or more probe(s).

28. (canceled)

29. The method of claim 27, wherein the capture bead is coated with streptavidin.

30-32. (canceled)

33. A cDNA molecule encoding nucleic acid

34. (canceled)

35. (canceled)


SEQUENCE LISTING (2 sequences)

SEQ ID NO: 1 - protein, 325 aa, Artificial Sequence
  AAL Q101N
MPTEFLYTSKIAAISWAATGGRQQRVYFQDLNGKIREAQRGGDNPWTGGSSQNVIGEAKLFSPLAAVTWKSAQGIQIRVYCVNKDNILSEFVYDGSKWITGNLGSVGVKVGSNSKLAALQWGGSESAPPNIRVYYQKSNGSGSSIHEYVWSGKWTAGASFGSTVPGTGIGATAIGPGRLRIYYQATDNKIREHCWDSNSWYVGGFSASASAGVSIAAISWGSTPNIRVYWQKGREELYEAAYGGSWNTPGQIKDASRPTPSLPDTFIAANSSGNIDISVFFQASGVSLQQWQWISGKGWSIGAVVPTGTPAGWLEHHHHHHHHHH

SEQ ID NO: 2 - DNA, 748 bp, Artificial Sequence
  AAL Q101N
atggaccggcgggtcgagccagaatgtaatcggcgaagcaaagcttttttcgccactggctgctgtcacgtggaaaagtgctcagggcatacagatccgtgtttactgcgtcaataaggataacatcctctccgaatttgtgtatgacggttcgaagtggatcaccggaaacctgggcagtgtcggcgtcaaggtgggctccaattcgaagcttgctgcgcttcagtggggcggatctgagagcgcccccccaaaaatccgagtttactaccagaagagcaacggtagtgggagctcaatccacgagtatgtctggtcgggcaaatggacggctggcgcaagctttgggtcaacggtgccaggaacgggtatcggagccaccgccatcgggccaggtcgcctgaggatctactaccaggctactgacaacaagatccgtgagcactgttgggactccaacagttggtacgtgggggggttctcggccagcgcttccgccggcgtctccatcgcggcgatttcttggggcagtacacccaacatccgggtctactggcagaaaggtagggaggaattgtacgaggctgcctatggcggttcatggaacactcctggtcagatcaaggacgcatccaggcctacgccctcgttgccagacacctttattgctgcgaactcctcggggaacatcgacatctctgtgttcttccaactcgagcatcatcatcaccatcaccaccatcatcat